(19) United States
(12) Patent Application Publication
(10) Pub. No.: US 2025/0248597 A1
(43) Pub. Date: Aug. 7, 2025

(54) METHOD FOR FORMING AN IMAGE OF A BODY BY MEANS OF AN MRI DEVICE

(71) Applicant: Multiwave Technologies AG, Plan les Ouates (CH)

(72) Inventors: Lei Zhang, Marseille 13E Arrondissement Cedex 13 (FR); Marc Dubois, Marseille 13E Arrondissement Cedex 13 (FR); Gianfranco Andia Vera, Marseille 13E Arrondissement Cedex 13 (FR); Tryfon Antonakakis, Marseille 13E Arrondissement Cedex 13 (FR)

(21) Appl. No.: 18/855,088

(22) PCT Filed: May 11, 2023
(86) PCT No.: PCT/EP2023/062618

§ 371 Date: Oct. 8, 2024

(30) Foreign Application Priority Data
May 17, 2022 (FR) FR2204672

(51) Int. Cl. A61B 5/00; A61B 5/055; G01R 33/44; G01R 33/56; G06T 7/00

(52) U.S. Cl. CPC A61B 5/0033; A61B 5/055; G01R 33/445; G01R 33/5608; G06T 7/0012; G06T 2207/10088

(57) ABSTRACT

A method for imaging a body, implements a basic sequence that includes:—generating at least one echo signal of a sequence of RF pulses produced by an RF coil;—performing an echo measurement of the at least one echo signal by the RF coil; and—performing a background measurement by the RF coil representative of a background signal. The method includes the following steps: a) repeating the basic sequence as many times as necessary so as to form a Fourier image of the body and a Fourier image of the background from the background measurements; and b) processing the Fourier image of the body and of the background so as to obtain an image of the body in which a signature of the one or more background signals is reduced or even eliminated.

### CROSS-REFERENCE TO RELATED APPLICATIONS

This application is a national phase entry under 35 U.S.C. § 371 of International Patent Application PCT/EP2023/062618, filed May 11, 2023, designating the United States of America and published as International Patent Publication WO 2023/222508 A1 on Nov. 23, 2023, which claims the benefit under Article 8 of the Patent Cooperation Treaty of French Patent Application Serial No. FR2204672, filed May 17, 2022.

### TECHNICAL FIELD

The present disclosure relates to the field of magnetic resonance imaging. In particular, the present disclosure relates to a method for forming an MRI image of a body using an MRI device.

### BACKGROUND

Magnetic resonance imaging (MRI) is currently widely used to image, non-invasively, the interior of bodies and, in particular, human bodies. In particular, magnetic resonance imaging makes it possible to probe the hydrogen nuclei, and, in particular, their nuclear spin, of water molecules forming part of the body.

In this respect, an MRI apparatus is provided with a magnet intended to impose on the body a static magnetic field (called “main magnetic field”), under the effect of which the nuclear spins associated with the hydrogen nuclei contained in the water molecules forming part of this body polarize.

In particular, the magnetic moments associated with these spins are preferentially aligned along an axis called the z axis, determined by the orientation of the main magnetic field so as to create a magnetization of the body.

An MRI apparatus also comprises gradient coils configured to produce magnetic fields of small amplitude and varying in space when a current is applied thereto. More particularly, the gradient coils are designed to produce a magnetic field component that is aligned parallel to the main magnetic field, and that varies linearly in amplitude with the position along one of the axes x, y or z (with each pair of axes x, y and z being perpendicular).

Thus, the combined effects of the magnetic fields imposed by the gradient coils make it possible to spatially encode each of the positions of the body intended to be probed.

An MRI apparatus also comprises at least one radiofrequency (RF) coil intended to act as an RF receiver transmitter. In particular, the at least one radio frequency coil is configured to emit RF energy pulses of a frequency equal to or close to the resonant frequency of the hydrogen nuclei spins and that is at least partially absorbed by these nuclei.

As soon as the RF emission is interrupted, the nuclear spins relax to return to their initial energy state and in turn emit an RF signal capable of being collected by at least one RF coil. This RF signal is then processed using a computer and reconstruction algorithms to obtain an image of the body.

The main magnetic field, generally comprised between 1.5 Tesla and 3 Tesla, makes it possible to achieve relatively reasonable signal-to-noise ratios and consequently to form images of the human body of sufficient quality and over durations on the order of one minute or more.

However, there are circumstances wherein it is not possible to implement a main magnetic field of such an intensity. Portable MRI apparatuses are an example thereof. The latter generally comprise a permanent magnet or electromagnets of limited capacity, and cannot impose a main magnetic field with an intensity greater than 60 mT, or even greater than 200 mT, without adversely affecting the mass or bulk of the MRI apparatus considered.

This limitation in terms of main magnetic field intensity directly affects the performance of the MRI apparatus, and the images obtained are imprinted with noise. That noise comprises two components, one correlated, the other uncorrelated.

The uncorrelated component is, in this respect, representative of a noise that depends essentially on the environment wherein the MRI device is located. The contribution of this component to an MRI image can be minimized by techniques known to the skilled person.

The correlated component, meanwhile, has a signature that may mask certain details in the images obtained by the MRI device in question. U.S. Pat. No. 9,797,971 B2 deals with this aspect, and thus proposes an MRI device configured to eliminate the correlated component. In particular, the device proposed in U.S. Pat. No. 9,797,971 B2 comprises, in addition to an RF coil dedicated to MRI measurement, a secondary coil for evaluating correlated noise that may be emitted and experienced by the MRI device. The secondary coil, as described in U.S. Pat. No. 9,797,971 B2, is the subject of a compromise in terms of positioning with respect to the RF coil. In particular, the secondary coil is positioned at a distance from the body being measured, so as to remain insensitive to the signal emitted by the body, but close enough to the RF coil to detect noise there of the same nature.

However, the MRI device proposed in U.S. Pat. No. 9,797,971 B2 is not satisfactory.

This is because that device does not allow correlated noise to be reduced without the consideration of approximations, which in turn affect the quality of the images obtained. Indeed, this document proposes to neglect the non-correlated component, which is nevertheless detected by the secondary coil, to establish a transfer function between the RF coil and the secondary coil.

Furthermore, insofar as the secondary coil is positioned to detect a noise of the same nature as that to which the RF coil is subjected, the secondary coil is also, and necessarily, sensitive to the signal emitted by the body. This last aspect, not discussed in U.S. Pat. No. 9,797,971 B2, can be a source of error on the MRI image of the body.

One aim of the present disclosure is to provide a method for forming an image of a body using a magnetic resonance imaging device that reduces, or even eliminates, any correlated noise signature.

### BRIEF SUMMARY

The present disclosure relates to a method for imaging a body by means of an MRI device, the method implementing a basic sequence of a repetition time TR that comprises:
generating at least one echo signal, over an echo time range PE, by subjecting the body positioned in an examination volume of the MRI device to a sequence of RF pulses produced by an RF coil, as well as to magnetic field gradients produced by gradient coils;
performing an echo measurement of the at least one echo signal by the RF coil;
performing a background measurement, by the RF coil, over a time range known as the background range PF, distinct from the echo range PE, and representative of a background signal;
the method comprising the following steps:
   a) repeating the basic sequence as many times as necessary to form, in Fourier space, a Fourier image of the body from the echo signals and a Fourier image of the background from the background measurements;
   b) a step for processing either the Fourier image of the body and the Fourier image of the background, or the echo signals and the background signals, so as to obtain an image of the body in real space, wherein a signature of the background signal(s) is reduced or even eliminated.

Implementing the present disclosure thus makes it possible to obtain an image of the body in real space that is essentially devoid of a signature of the background signal(s).

In one embodiment, step b) comprises processing the Fourier image of the body and the Fourier image of the background, step b) being performed in two sub-steps b1) and b2),
step b1) comprising mathematical processing of the background and body Fourier images to subtract the background signal from the body Fourier image to form a processed Fourier image in Fourier space,
and step b2) comprising a transformation of the processed Fourier image to obtain an image of the body in real space.

According to one embodiment, the mathematical processing step b1) comprises at least one of the following methods: spectral subtraction method, anisotropic diffusion filtering, non-local means.

According to one embodiment, step b) is performed in two sub-steps b3) and b4),
step b3) comprising a transformation of the Fourier image of the body and the Fourier image of the background into, respectively, an image of the intermediate body and an image of the background in real space,
step b4) comprising a processing of the intermediate body image and the background image to subtract the contribution of the background signal in the intermediate body image to obtain an image of the body in real space.

According to one embodiment, step b4) comprises processing by principal component analysis.

According to one embodiment, the generating of at least one echo signal comprises the implementation of an electromagnetic pulse, known as the initial electromagnetic pulse, orthogonal to a permanent magnetic field Bo imposed on the body located in the examination volume of the MRI device during the repetition of the basic sequence.

According to one embodiment, the initial electromagnetic pulse is followed by at least one electromagnetic rephasing pulse and concomitant with the selection of a slice by way of one of the gradient coils referred to as the “slice plane selection coil,” while the measurement of the at least one echo signal implements phase and frequency encoding by two of the gradient coils referred to as a “phase gradient coil” and a “frequency gradient coil.”

According to one embodiment, the basic sequence is a spin echo sequence that comprises a single measurement of an echo signal, wherein the background measurement is performed after the measurement of the echo signal.

According to one embodiment, the background measurement is performed with phase and frequency coding identical to the phase and frequency coding implemented during the echo signal measurement.

According to one embodiment, the basic sequence is a fast spin echo sequence that comprises repeating a sub-sequence within the sequence, the sub-sequence comprising the electromagnetic rephasing pulse and the measurement of an echo signal.

According to one embodiment, the basic sequence is a 3D fast spin echo sequence that comprises repeating N basic sub-sequences within the sequence, each basic sub-sequence comprising the electromagnetic rephasing pulse and the measurement of an echo signal.

According to one embodiment, the basic sequence comprises a single background measurement, advantageously performed after the basic sub-sequences.

According to one embodiment, the basic sequence comprises N background measurements, each background measurement being performed within its own basic sub-sequence.

According to another embodiment, a computer program comprises instructions that, when the program is executed by a computer, implements the steps of the method according to the present disclosure.

An MRI device may be provided with a unit configured to implement the computer program according to the present disclosure, and the MRI device may comprise the computer program.

### BRIEF DESCRIPTION OF THE DRAWINGS

Other features and advantages of the present disclosure will emerge from the following detailed description of an example embodiment of a body imaging method according to the present disclosure, with reference to the appended figures, wherein:

FIG. 1 is a schematic representation according to an exploded view of a magnetic resonance imaging device;

FIG. 2 is a schematic representation of a fast 3D spin echo sequence that may be considered for implementing the MRI imaging process according to the present disclosure;

FIG. 3 is a representation of a first example of a fast 3D spin echo sequence from FIG. 2;

FIG. 4 is a Fourier image of a body;

FIG. 5 is a Fourier image of the background;

FIG. 6 is an image of the body obtained without using a method according to the present disclosure; and

FIG. 7 is an image of a body in real space.

### DETAILED DESCRIPTION

The present disclosure relates to a method for forming an image of a body using a magnetic resonance imaging (hereinafter “MRI”) device.

More particularly, the present disclosure relates to a method for forming an image of a body arranged in an examination volume of the MRI device. In this respect, it is well known that such a device is subject to environmental noise, hereinafter referred to as a “background signal.” This background signal, generally associated with surrounding electromagnetic signals, can have a negative impact on the performance of the MRI device. More specifically, this background signal may present a signature on the images of the body that can be obtained with an MRI device, and, in particular, may conceal its details.

To overcome this drawback, MRI devices are generally isolated by way of special shielding (e.g., Faraday cages). However, this solution remains unattractive when considering a portable MRI device.

To this end, the present disclosure relates to a method for imaging a body using an MRI device, the method implementing a basic sequence of a repetition time TR that comprises:
generating at least one echo signal, over an echo time range PE, by subjecting the body positioned in an examination volume of the MRI device to a sequence of RF pulses produced by an RF coil, as well as to magnetic field gradients produced by gradient coils;
performing an echo measurement of the at least one echo signal by the RF coil;
performing a background measurement, by the RF coil, over a time range known as the background range PF, distinct from the echo range PE, and representative of a background signal;
the method comprising the following steps:
   a) repeating the basic sequence as many times as necessary to form, in Fourier space, a Fourier image of the body from the echo signals and a Fourier image of the background from the background measurements;
   b) a step for processing either the Fourier image of the body and the Fourier image of the background, or the echo signals and the background signals, so as to obtain an image of the body in real space, essentially devoid of a signature of the background signal(s).

For instance, and according to a first alternative, step b) comprises processing the Fourier image of the body and the Fourier image of the background, step b) being performed in two sub-steps b1) and b2),
Step b1) comprises mathematical processing of the background and body Fourier images to subtract the background signal from the body Fourier image to form a processed Fourier image in Fourier space,
Step b2) comprises a transformation of the processed Fourier image to obtain an image of the body in real space.

According to a second alternative, step b) is performed in two sub-steps b3) and b4)
Step b3) comprises a transformation of the Fourier image of the body and the Fourier image of the background into, respectively, an image of the intermediate body and an image of the background in real space,
Step b4) comprises a processing of the intermediate body image and the background image to subtract the contribution of the background signal in the intermediate body image to obtain an image of the body in real space.

It is understood that the MRI imaging process according to the terms of the present disclosure is particularly suitable for use with a low-field MRI device (e.g., a portable MRI device), i.e., an MRI device operating with a main magnetic field of less than 0.5 Tesla, or even less than 0.2 Tesla. Nevertheless, the present disclosure need not be limited to this aspect. In particular, the person skilled in the art will be able to consider the implementation of the method according to the present disclosure to operate a high-field MRI device.

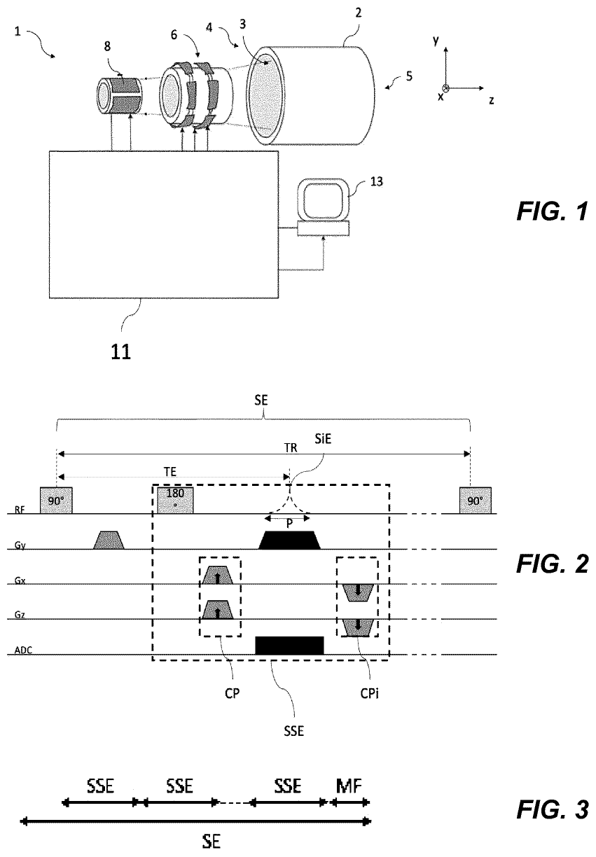

FIG. 1 is a representation of an MRI device 1 suitable for implementing the imaging method according to the present disclosure.

The MRI device 1 comprises a magnet 2 configured to impose a main magnetic field Bo. The magnet 2 may, for example, comprise a permanent magnet. The magnet 2 may, in particular, extend along an elongation axis z.

More particularly, the magnet 2 defines a bore 3 that opens up through a first opening 4 and a second opening 5 opposite each other along the elongation axis z.

In this respect, the magnet 2 is arranged to allow the insertion of a body, and more particularly of a human body, into the bore 3 through the first opening 4 along the elongation axis z.

The magnet 2 may be configured to impose a principal magnetic field Bo oriented along an axis perpendicular to the elongation axis z, in a zone referred to as the “analysis zone,” of the bore 3.

In this respect, the magnet 2 may comprise an assembly of basic magnets, and, in particular, arranged in series of Halbach rings. European Patent No. 3368914 B1 gives an example thereof. However, the invention is not limited solely to the configuration described in European Patent No. 3368914 B1.

By way of example, the magnet 2 is configured to impose a principal magnetic field with an amplitude of less than 0.1 Tesla, advantageously less than 0.065 Tesla, even more advantageously less than or equal to 0.05 Tesla.

The MRI device 1 also comprises a set of gradient coils 6. The gradient coils 6 are particularly configured to produce small-amplitude magnetic fields and vary in space when a current is applied thereto.

More particularly, the gradient coils 6 are designed to produce a magnetic field component that is aligned parallel to the principal magnetic field, and that varies linearly in amplitude with the position along one of the axes x, y or z (the axes x, y and z forming an orthogonal reference frame).

Thus, the combined effects of the magnetic fields imposed by the gradient coils 6 make it possible to spatially encode the signals coming from a body present in the bore 3 and intended to be probed. Spatial encoding is manifested, in particular, by a variation in the resonance energy of the nuclear spins of the hydrogen nuclei comprised in the body intended to be probed and present in the analysis zone. In other words, the nuclear spins of the hydrogen nuclei are subjected to a magnetic field that differs from one position to another.

The MRI device 1 further comprises a radio-frequency coil 8 (hereinafter “RF coil”). In particular, the RF coil 8 is arranged in the bore 3 and delimits an examination volume of the MRI device 1, wherein a body is intended to be housed.

Finally, the MRI device 1 further comprises means for controlling the MRI device 1. In particular, these control means may comprise a computer 13 interfaced, via interface means 11, with the various elements forming the MRI device 1.

The MRI device 1 described above can be used to carry out the method for forming an MRI image of a body located in the examination volume, in accordance with the present disclosure.

Under the terms of the present disclosure, a body image can be two-dimensional or three-dimensional.

Thus, the method for image formation according to the present disclosure comprises the implementation of a basic sequence SE. In particular, the basic sequence SE can be of a duration referred to as the repetition duration TR.

Thus, FIG. 2 is a schematic representation of a basic sequence SE that may be considered when carrying out the method according to the present disclosure. In particular, the basic sequence SE shown in FIG. 2 is a Turbo Spin Echo 3D sequence. However, the invention is not limited to this single sequence, and the person skilled in the art will be able to adapt its principles to other types of sequences. For example, a basic spin echo sequence SE, a fast spin echo sequence, may be considered for use in the present disclosure.

Thus, the basic sequence SE according to the present disclosure can comprise the generating of at least one echo signal SiE, over an echo time range PE, by subjecting the body to be imaged, positioned in an examination volume of the MRI device, to a sequence of RF pulses. In particular, RF pulses are generated by RF coil 8. Gradient coils (Gx, Gy and Gz) are used to spatially encode the body in the examination volume.

Note that the gradient coils are also used, or powered, throughout the execution of a measurement sequence (in particular, during the execution of the basic sequence(s)) to homogenize the main magnetic field B₀ to which the body is subjected in the analysis volume. In other words, the gradient coils produce local fields that are added to the main magnetic field B₀, so that the resultant field is more homogeneous than the main magnetic field B₀ in the analysis volume.

The basic sequence SE shown in FIG. 2 begins with the emission of an RF pulse, known as the main electromagnetic pulse, designed to excite the entire body volume. In some situations, the main electromagnetic pulse is referred to as a 90° pulse, in particular, when the pulse in question is in a plane orthogonal to the main magnetic field B₀.

The basic sequence SE also comprises N (N integer strictly greater than 1) basic sub-sequences SSE. In particular, the N basic sub-sequences are performed successively and following the emission of the main electromagnetic pulse.

Each basic sub-sequence SSE comprises, performed in succession:
(i) the emission of an RF pulse, known as a rephasing pulse, by the RF coil;
(ii) phase encoding referred to as “CP,” performed by means of gradient coils Gx and Gz to select a Ky line in Fourier space (also known as K space).
(iii) concomitant echo measurement of a SE echo signal by the RF coil and frequency coding by the Gy gradient coil.

Optionally, the basic sub-sequence SSE may comprise a step (iv) performed after the step (iii) to invert the phase encoding (CPi in FIG. 2) performed by the gradient coils Gx and Gz.

The step (iii) makes it possible to fill a Ky frequency line in K-space for a given phase encoding.

It is understood that echo measurement by the RF coil involves interfacing means, such as an analog-to-digital converter (ADC). In particular, measurement by the RF coil requires activation of the analog/digital converter. When no measurement is required, the analog/digital converter is disabled.

During the course of a basic sequence SE, the spin echo signals SiE occur at specific times, known as echo times TE. In particular, and starting from the moment of emission of the main electromagnetic pulse, the SiE spin echo signals are regularly spaced from one another by an echo time TE. In addition, each spin echo is spread over the PE echo time range.

In addition, each basic sub-sequence SSE has its own phase encoding. In other words, performing a basic sequence SE fills as many Ky lines in the K space as there are basic sub-sequences SSE in the basic sequence SE. In particular, performing a basic sequence fills N different Ky lines in K space.

According to the present disclosure, the basic sequence SE comprises at least one background measurement MF, by the RF coil, over a so-called background time range PF distinct from the echo time range PE, and representative of a background signal.

Considering a background time range PF distinct from the echo time range PE (in other words, these two ranges do not overlap) enables the RF coil to measure only the environmental signal, devoid of any spin echo likely to originate from the body.

A background measurement can be performed at any time distinct from the echo times. In particular, the background measurement, which is implemented by the RF coil, can take place before or after a spin echo measurement.

According to a first example shown in FIG. 3, the MF background measurement can be performed following the SSE basic sub-sequences. In particular, the background measurement MF implements frequency coding by means of the Gy gradient coil. This background measurement MF is used to obtain a frequency coding line. This line is consequently used to fill, in K space, as many Ky lines as there are basic sub-sequences SSE in the basic sequence SE.

Alternatively, and according to a second example, the basic sequence SE comprises a plurality of background measurements MF. In particular, the basic sequence SE comprises as many background measurements MF as there are basic sub-sequences SSE. In particular, and according to this second example, each sub-sequence SSE is associated with a background measurement. For example, and without limiting the present disclosure to this aspect, the background measurement can include Gx and Gz phase coding identical to that imposed when executing the basic sub-sequence with which it is associated.

Whatever the example considered, the background measurement(s) FM is/are performed over a time range distinct from the echo time ranges.

The method according to the present disclosure thus comprises a step a) of repeating the basic sequence as many times as necessary to form, in Fourier space, a Fourier image of the body from the echo signals and a Fourier image of the background from the background measurements.

Step a) is followed by a step b) of processing either the Fourier image of the body and the Fourier image of the background, or the echo signals and the background signals, so as to obtain an image of the body in real space, essentially devoid of a signature of the background signal(s).

For instance, and according to the first alternative, step b) comprises processing the Fourier image of the body and the Fourier image of the background, step b) being performed in two sub-steps b1) and b2).

Step b1) comprises mathematical processing of the background and body Fourier images to subtract the background signal from the body Fourier image and to form a processed Fourier image in Fourier space.

The mathematical processing carried out in step b1) may comprise at least one of the following methods: spectral subtraction method, anisotropic diffusion filtering, non-local means.

The spectral subtraction method is described in M Arcan et al.: “Denoising MRI using spectral subtraction.” IEEE transactions on bio-medical engineering vol. 60 (6): 1556-1562 (2013).

The skilled person wishing to implement anisotropic diffusion filtering may consult the document Perona P, Malik J.: “Scale-space and edge-detection using anisotropic diffusion,” IEEE Trans Pattern Anal Mach Intell. July; vol. 12 (7): 629-639 (1990).

A description of non-local means can be found in Buades A et al, “Non-local algorithm for image denoising,” Proc IEEE Comput Soc Conf Comput Vis Pattern Recognit, vol. 2:60-65 (2005).

In general, it may be considered to perform step b1) according to a method belonging to at least one of the classes chosen from: Spectral subtraction method, Wiener-type method, subspace method, statistical model-based method.

For further information, please refer to Shrawankar, U., Thakare, V. (2010). Noise Estimation and Noise Removal Techniques for Speech Recognition in Adverse Environment. In: Shi, Z., Vadera, S., Aamodt, A., Leake, D. (eds) Intelligent Information Processing V. IIP 2010. IFIP Advances in Information and Communication Technology, vol 340. Springer, Berlin, Heidelberg.

Step b2) comprises a transformation of the processed Fourier image to obtain an image of the body in real space.

This transformation may comprise the use of a Fourier transform.

Alternatively, the invention can implement the transformation described in Usman M, Kakkar L et al, “Joint B0 and image estimation integrated with model based reconstruction for field map update and distortion correction in prostate diffusion MRI,” Magn Reson Imaging, vol. 65:90-99 (2020).

According to a second alternative, step b) is performed in two sub-steps b3) and b4).

Step b3) comprises a transformation of the Fourier image of the body and the Fourier image of the background into, respectively, an image of the intermediate body and an image of the background in real space,

This transformation may comprise the use of a Fourier transform.

Step b4) comprises a processing of the intermediate body image and the background image to subtract the contribution of the background signal in the intermediate body image to obtain an image of the body in real space.

Advantageously, step b4) comprises processing by principal component analysis.

This processing is described in Campbell-Washburn A E et al.; “Using the robust principal component analysis algorithm to remove RF spike artifacts from MR images” Magn Reson Med.; vol. 75 (6): 2517-2525 (2016).

The invention has been described with x, y, z coordinates (in relation to Gx, Gy, and Gz gradient coils). It is understood that the order of these coordinates has been chosen arbitrarily, so that the person skilled in the art may consider a different order.

The method according to the present disclosure thus makes it possible to eliminate the background signal of an MRI image. In particular, this method proposes to insert an additional event during the performance of a basic sequence of measurement of a background signal with the RF coil. Unlike the solution proposed in U.S. Pat. No. 9,797,971 B2, the method according to the present disclosure does not impose any approximations when performing step b).

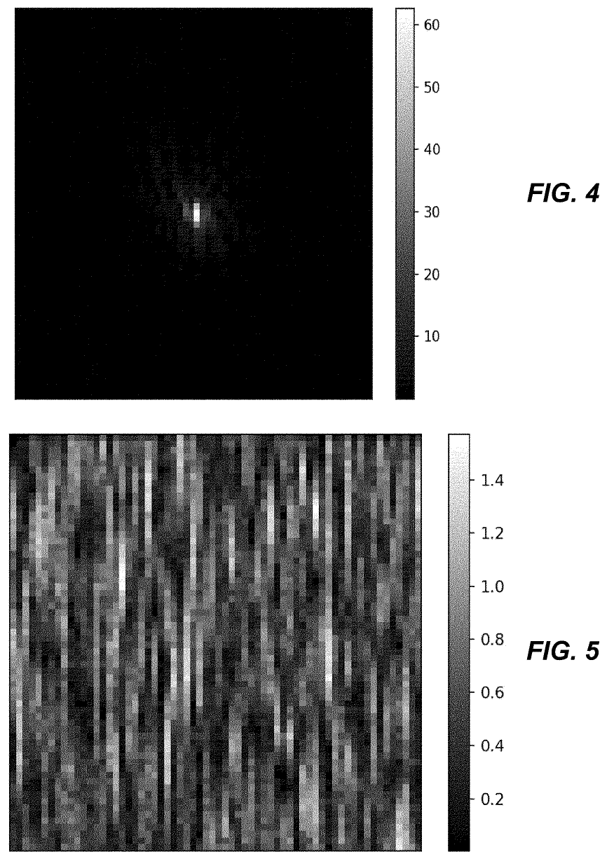

FIGS. 4-7 are illustrations of the implementation of the present disclosure according to the first alternative for imaging two samples E1 and E2.

In particular, FIG. 4 is a Fourier image of the body, while FIG. 5 is a Fourier image of the background.

By performing a mathematical processing step b1) on these two Fourier images of the background and the body, the background signal is subtracted from the Fourier image of the body and a processed Fourier image (not shown) is formed in Fourier space. The mathematical processing used in this example comprises a spectral subtraction method.

FIG. 7 shows the Fourier image of the body (the two samples E1 and E2), in real space, obtained by transforming the Fourier image. This image shows the two samples E1 and E2 and a line T representative of the background signal.

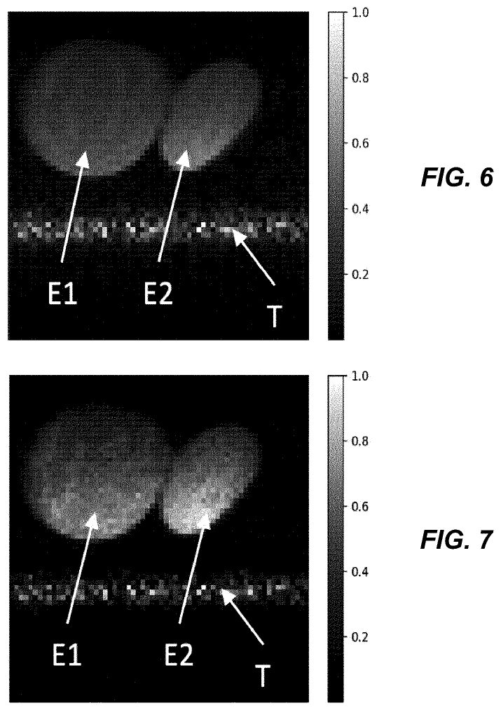

FIG. 6 is a representation of the body image in real space, with no mathematical processing to reduce or even eliminate the background signal signature. Comparing the images shown in FIGS. 6 and 7 reveals that the background signal signature is significantly reduced compared with the signal characterizing the body.

The present disclosure also relates to a computer program comprising instructions that, when the program is executed by a computer, implements the steps of the method according to the present disclosure.

The present disclosure also relates to an MRI device provided with a unit configured to implement the computer program according to the present disclosure, the MRI device comprising the computer program.

Of course, the present invention is not limited to the described embodiments and variant embodiments may be envisaged without departing from the scope of the invention as defined by the claims.

1. A method for imaging a body using an MRI device, the method implementing a basic sequence of a repetition time TR that comprises:
generating at least one echo signal over an echo time range PE by subjecting the body positioned in an examination volume of the MRI device to a sequence of RF pulses produced by an RF coil and to magnetic field gradients produced by gradient coils;
performing an echo measurement of the at least one echo signal by the RF coil; and
performing a background measurement, by the RF coil, over a background time range PF distinct from the echo range PE and representative of a background signal;
the method comprising the following steps:
a) repeating the basic sequence as many times as necessary to form, in Fourier space, a Fourier image of the body from the echo signals and a Fourier image of the background from the background measurements; and
b) a step for processing either the Fourier image of the body and the Fourier image of the background, or the echo signals and the background signals, so as to obtain an image of the body in real space, wherein a signature of the background signal(s) is reduced or even eliminated.

2. The method of claim 1, wherein step b) comprises processing the Fourier image of the body and the Fourier image of the background, step b) being performed in two sub-steps b1) and b2),
wherein step b1) comprises mathematical processing of the background and body Fourier images to subtract the background signal from the body Fourier image to form a processed Fourier image in Fourier space, and
wherein step b2) comprising a transformation of the processed Fourier image to obtain an image of the body in real space.

3. The method of claim 2, wherein the mathematical processing step b1) comprises at least one of the following methods: spectral subtraction method, anisotropic diffusion filtering, or non-local means.

4. The method of claim 1, wherein step b) is performed in two sub-steps b3) and b4),
wherein step b3) comprises a transformation of the Fourier image of the body and the Fourier image of the background into, respectively, an image of the intermediate body and an image of the background in real space, and
wherein step b4) comprises a processing of the intermediate body image and the background image to subtract the contribution of the background signal in the intermediate body image to obtain an image of the body in real space.

5. The method of claim 4, wherein step b4) comprises processing by principal component analysis.

6. The method of claim 1, wherein the generating of the at least one echo signal comprises implementing an initial electromagnetic pulse orthogonal to a permanent magnetic field B₀ imposed on the body located in the examination volume of the MRI device during the repetition of the basic sequence.

7. The method of claim 6, wherein the initial electromagnetic pulse is followed by at least one electromagnetic rephasing pulse and concomitant with the selection of a slice by way of one of the gradient coils comprising a slice plane selection coil, while the measurement of the at least one echo signal implements phase and frequency encoding by two of the gradient coils
comprising a phase gradient coil and a frequency gradient coil.

8. The method of claim 7, wherein the basic sequence is a spin echo sequence comprising a single measurement of an echo signal, wherein the background measurement is performed after the measurement of the echo signal.

9. The method of claim 8, wherein the background measurement is performed with phase and frequency coding identical to a phase and frequency coding implemented during the echo signal measurement.

10. The method of claim 7, wherein the basic sequence is a fast spin echo sequence comprising repeating a sub-sequence within the sequence, the sub-sequence comprising the electromagnetic rephasing pulse and the measurement of an echo signal.

11. The method of claim 6, wherein the basic sequence is a 3D fast spin echo sequence comprising repeating N basic sub-sequences within the sequence, each basic sub-sequence comprising the electromagnetic rephasing pulse and the measurement of an echo signal.

12. The method of claim 11, wherein the basic sequence comprises a single background measurement.

13. The method of claim 11, wherein the basic sequence comprises N background measurements, each background measurement being performed within its own basic sub-sequence.

14. An MRI device provided with a unit on which a computer program is loaded, the MRI device being configured to implement the method according to claim 1.

15. The method of claim 12, wherein the single background measurement is performed after the basic sub-sequences.
